SEQUENCE LISTING (1 sequence)

SEQ ID NO: 1 - protein, 36 aa, Artificial Sequence
  synthetic
EQSDFCPWYTGLPFIPYLDNLPNFNRSIDGPIRLPI

(19) United States
(12) Patent Application Publication
(10) Pub. No.: US 2021/0382055 A1
(43) Pub. Date: Dec. 9, 2021

(54) USES OF 4210 DA PEPTIDE AS A MARKER IN DIAGNOSIS OF LIVER CANCER AND CIRRHOSIS

(71) Applicant: Hebei Medical University, Shijiazhuang (CN)

(72) Inventors: Dianwu LIU, Shijiazhuang (CN); Yuanyuan REN, Shijiazhuang (CN); Man LI, Shijiazhuang (CN)

(21) Appl. No.: 16/894,796

(22) Filed: Jun. 6, 2020

(51) Int. Cl. G01N 33/576; G01N 33/68; G01N 33/574

(52) U.S. Cl. CPC G01N 33/5761; G01N 2800/085; G01N 33/57438; G01N 33/6851

(57) ABSTRACT

Disclosed herein are uses of 4210 peptide as a marker in the diagnosis of liver cancer and cirrhosis. The 4210 Da peptide is differentially expressed in the serum samples of subjects with different hepatitis B-associated liver diseases, specifically, the expression level is the highest in patients with chronic hepatitis B, sequentially followed by patients with cirrhosis and hepatocellular carcinoma, and healthy controls and those with natural clearance of hepatitis B virus have the lowest expression level of the 4210 Da peptide. The invention provides a novel marker for assisting the diagnosis of a hepatitis B-associated liver disease, which can effectively assist the diagnosis of the hepatocellular carcinoma and the cirrhosis developed from chronic hepatitis B, benefiting the early diagnosis of hepatitis B-associated liver diseases.

### REFERENCE TO AN ELECTRONIC SEQUENCE LISTING

The contents of the electronic sequence listing (Untitled ST25.txt; Size: 1,000 bytes; and Date of Creation: Aug. 17, 2020) is herein incorporated by reference in its entirety.

### TECHNICAL FIELD

This application relates to medical diagnosis, and more particularly to uses of 4210 Da as a marker in the diagnosis of liver cancer and cirrhosis.

### BACKGROUND

Hepatocellular carcinoma (HCC) is the most frequent primary liver cancer around the world. In China, hepatocellular carcinoma is the second death cause only following lung cancer among the cases suffering from malignant tumors, and there are about 323,000 cases died from primary liver cancer every year. It has been found that more than 80% of the cases with primary liver cancer are associated with HBV infection and have suffered from cirrhosis before. The development stages of the primary liver cancer can be divided into chronic viral infection, cirrhosis and liver cancer according to the clinical pathological characteristics. Since the early symptoms of HCC are not obvious, it is often clinically diagnosed at the later stage. Therefore, it is of great significance to develop an appropriate marker to achieve the early diagnosis for HCC.

### SUMMARY

An object of this application is to provide applications of a 4210 Da peptide as a marker in the diagnosis of liver cancer and cirrhosis.

The invention provides a method of diagnosing a hepatitis B-associated liver disease in a subject, comprising:

(1) determining an abundance of a 4210 Da peptide in a serum sample from the subject using a matrix-assisted laser desorption ionization time-of-flight mass spectrometer; and

(2) diagnosing the hepatitis B-associated liver disease according to the abundance of the 4210 Da peptide in the serum sample.

In an embodiment, in step (2), the diagnosis is performed as follows:

(a1) when 4210 Da peptide is used to discriminate HBV-related cirrhosis/hepatocellular carcinoma from chronic hepatitis, then if the abundance of the 4210 Da peptide in the serum sample is greater than or equal to 86.5 and less than 233.5, the hepatitis B-associated liver disease is cirrhosis or hepatocellular carcinoma, otherwise, the hepatitis B-associated disease is chronic hepatitis B.

In an embodiment, in step (2), the diagnosis is performed as follows:

(b1) when 4210 Da peptide is used to discriminate HBV-related cirrhosis from normal subject, then if the abundance of the 4210 Da peptide in the serum sample is greater than or equal to 96, the subject suffers from cirrhosis, otherwise, the subject is a normal subject;

(b2) when 4210 Da peptide is used to discriminate HBV-related hepatocellular carcinoma from normal subject, then if the abundance of the 4210 Da peptide in the serum sample is greater than or equal to 86.5, the subject suffers from hepatocellular carcinoma, otherwise, the subject is a normal subject;

(b3) when 4210 Da peptide is used to discriminate HBV-related cirrhosis from chronic hepatitis, then if the abundance of the 4210 Da peptide in the serum sample is less than 232.50, the hepatitis B-associated liver disease is cirrhosis, otherwise, the hepatitis B-associated liver disease is chronic hepatitis B; and

(b4) when 4210 Da peptide is used to discriminate HBV-related hepatocellular carcinoma from chronic hepatitis, then if the abundance of the 4210 Da peptide in the serum sample is less than 233.5, the hepatitis B-associated liver disease is hepatocellular carcinoma, otherwise, the hepatitis B-associated liver disease is chronic hepatitis B.

In an embodiment, accuracies of (b1), (b2), (b3) and (b4) are 0.816, 0.750, 0.685 and 0.712, respectively.

In an embodiment, an amino acid sequence of the 4210 Da peptide is shown as SEQ ID NO:1.

In an embodiment, the abundance of the 4210 Da peptide in a serum sample refers to a relative abundance of the 4210 Da peptide in the serum sample.

Compared to the prior art, the invention has the following beneficial effects.

The invention provides a novel marker for assisting the diagnosis of a hepatitis B-associated liver disease, which can effectively assist the diagnosis of hepatocellular carcinoma developed from chronic hepatitis B and cirrhosis developed from chronic hepatitis B. Therefore, the invention is of great significance for the early diagnosis of the hepatitis B-associated liver diseases.

### BRIEF DESCRIPTION OF THE DRAWINGS

FIG. 1 is a box plot showing the abundance of the 4210 Da peptide in serum samples from respective groups.

FIG. 2 shows an ROC curve of the diagnosis of hepatocellular carcinoma from chronic hepatitis B in the use of the 4210 Da peptide as a molecular marker.

FIG. 3 shows an ROC curve of the diagnosis of cirrhosis from chronic hepatitis B in the use of the 4210 Da peptide as a molecular marker.

FIG. 4 shows an ROC curve of the diagnosis of hepatocellular carcinoma from healthy controls in the use of the 4210 Da peptide as a molecular marker.

FIG. 5 shows an ROC curve of the diagnosis of cirrhosis from healthy controls in the use of the 4210 Da peptide as a molecular marker.

### DETAILED DESCRIPTION OF EMBODIMENTS

The invention will be described in detail below with reference to the embodiments to make the technical solutions more understandable, and these embodiments are not intended to limit the invention. Unless otherwise specified, the experiments in the embodiments are performed in a conventional manner, and the reagents used therein are conventional and commercially available. The qualitative tests performed in the embodiments each are repeated three times, and the results are expressed as mean.

130 serum samples from healthy controls (HC), 50 serum samples from cases with natural clearance of hepatitis B viruses (NC, for short), 130 serum samples from patients with chronic hepatitis B (CHB), 130 serum samples from patients with liver cirrhosis (LC) and 130 serum samples from patients with hepatocellular carcinoma (HCC) are collected, where there is no significant significance in the age and gender of subjects among the groups. The liver cirrhosis and the hepatocellular carcinoma are both developed from the chronic hepatitis B. The diagnosis is performed in accordance with the criteria in “Guidelines for the Prevention and Treatment of chronic hepatitis B” (version 2010), which are jointly developed by Chinese Society of Infectious Diseases and Chinese Society of Hepatology of Chinese Medical Association. The inclusion criteria include: (1) Han nationality from the northern China; (2) no limit in the gender and age; and (3) the subject should be informed and agree to receive the test. The exclusion criteria include: (1) acute or chronic hepatitis caused by other types of hepatitis (such as hepatitis A, hepatitis C and hepatitis D) viruses and infection caused by human immunodeficiency virus; (2) liver damage caused by alcohol, autoimmunity, drugs, parasites and other microorganisms; (3) cases with acute hepatitis B and non-HBV-associated liver cancer; and (4) those who cannot participate or are unwilling to sign the informed consent form.

In the embodiments, peptides in a serum sample are extracted using a peptide-extracting magnetic bead kit (SPE-C), which is purchased from Beijing YixinBochuang Biotechnology Co., Ltd. The kits are operated as recommended, and the resulting products are directly analyzed by matrix-assisted laser desorption ionization time-of-flight mass spectrometry.

Matrix-assisted laser desorption ionization time-of-flight mass spectrometry is a novel soft ionization biological mass spectrometry detection method, which has made a great progress in recent years, and is used below to analyze the abundance of the 4210 Da peptide in the serum samples. The matrix-assisted laser desorption ionization time-of-flight mass spectrometer employed herein is a Clin-TOF mass spectrometer, which is also manufactured by Beijing YixinBochuang Biotechnology Co., Ltd. The related detection parameters are set as follows: tuning mode: liner; power: 70 mV; profiles: 50; shots: 10; max laser rep rate: 30 m/z; and range: 100-10,000.

The Youden index refers to the sum of sensitivity and specificity minus 1, which indicates the characteristic of the screening method to distinguish real patients from non-patients, that is, a larger index indicates better effect of the screening experiment and higher authenticity. The sensitivity, also referred to as true positive rate (TPR), is calculated as follows: TPR=TP/(TP+FN); the specificity, also referred to as true negative rate (TNR), is calculated as follows: TNR=TN/(TN+FP); and the accuracy (ACC) is calculated as follows: ACC=(TP+TN)/(TP+FP+FN+TN), where TP: true positive; TN: true negative; FP: false positive; and FN: false negative.

### Example 1 Demonstration of 4210 Da Peptide as a Diagnostic Marker

The inventor, through the matrix-assisted laser desorption ionization time-of-flight mass spectrometry analysis, had found a peptide, i.e., the 4210 Da peptide, in the process of seeking for a potential serum biological marker for the early diagnosis of liver cirrhosis and hepatocellular carcinoma.

The 4210 Da peptide was identified to be a 36-amino acid fragment of eukaryotic peptide release factor 3b (eRF3b), and had a sequence of EQSDFCPWYTGLPFIPYLDNLPNFNRSIDGPIRLPI (SEQ ID NO: 1).

It has been further demonstrated that the expression of the 4210 Da peptide varied with the development of HBV-associated liver diseases, indicating the applicability of the 4210 Da peptide as a biological marker to the diagnosis of cirrhosis and liver cancer. As a diagnostic marker, the 4210 Da peptide had good sensitivity and specificity, especially in the development stage from chronic hepatitis B to cirrhosis and liver cancer, so it was of special significance for the 4210 Da peptide to be used as a serum biological marker for the early diagnosis of liver cancer, facilitating the timely intervention in the occurrence and development of liver cancer.

### Example 2 Diagnosis of Hepatitis B-Associated Cirrhosis and Hepatocellular Carcinoma with the 4210 Da Peptide

(1) Determination of an Abundance of the 4210 Da Peptide

Peptides in respective serum samples are extracted and analyzed by matrix-assisted laser desorption ionization time-of-flight mass spectrometry to obtain the abundance of the 4210 Da peptide in respective serum samples.

(2) Expression of the 4210 Da Peptide in Different Populations

The results of the abundance of 4210 Da peptides in serum samples from each group of people were shown in Table 1, and expressed as “median (quartile)”.

TABLE 1
Abundance of 4210 Da peptide in serum samples
from respective groups of people

| | 4210 Da (Intensity), M(QR) |
|---|---|
| HC(N = 130) | 38.00 (63.00) |
| NC (N = 50) | 32.50 (50.00) |
| CHB (N = 130) | 306.00 (215.00) |
| LC (N = 130) | 192.00 (192.00) |
| HCC (N = 130) | 145.50 (203.00) |
| P-value | <0.001 |

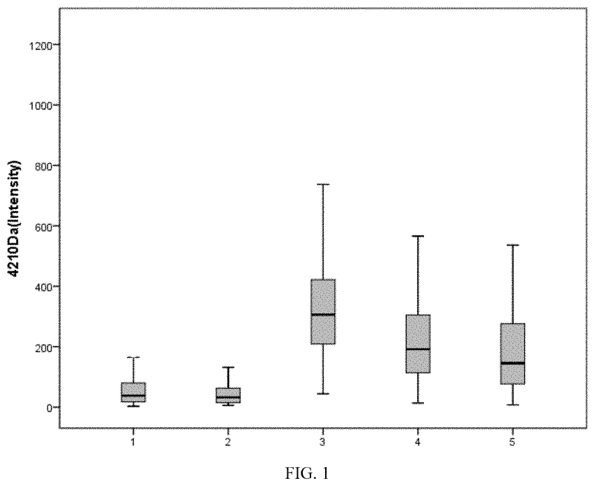

FIG. 1 is a box plot showing the abundance of the 4210 Da peptide in serum samples from respective groups. As shown in FIG. 1, 1-5 respectively corresponded to HC, NC, CHB, LC and HCC, and the ordinate indicated the intensity of the 4210 Da peptide.

It can be seen from the results that the intensity of the 4210 Da peptide in the serum sample from the patients with chronic hepatitis B was the highest, sequentially followed by patients with cirrhosis and patients with hepatocellular carcinoma, and healthy controls and those with natural clearance of hepatitis B virus were the lowest with respect to the intensity of the 4210 Da peptide.

(3) Diagnosis of Hepatocellular Carcinoma from Chronic Hepatitis B Using the 4210 Da Peptide as a Molecular Marker

The 130 serum samples from the patients with chronic hepatitis B and the 130 serum samples from the patients with hepatocellular carcinoma were analyzed herein.

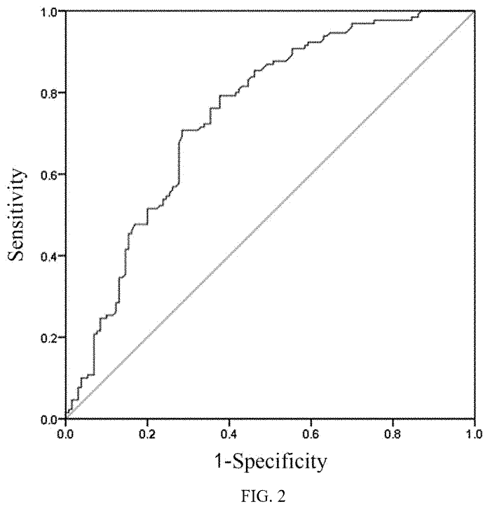

The abundance of the 4210 Da peptide in the serum sample was used as an indicator, and 233.50 was used as a threshold value, where a case can be determined to suffer from hepatocellular carcinoma when the abundance of the 4210 Da peptide was less than 233.5. Sensitivity, specificity, Youden index and accuracy of the diagnosis of hepatocellular carcinoma from chronic hepatitis B were shown in Table 2; the ROC curve was shown in FIG. 2; and the area under the ROC curve was shown in Table 3.

TABLE 2
Sensitivity, specificity, Youden index and accuracy of the diagnosis
of hepatocellular carcinoma from chronic hepatitis B

| Sensitivity | Specificity | Youden index | Accuracy |
|---|---|---|---|
| 0.715 | 0.708 | 0.423 | 0.712 |

TABLE 3
Area under the ROC curve of the diagnosis of hepatocellular
carcinoma from chronic hepatitis B

| | | | Asymp 95% confidence | |
|---|---|---|---|---|
| Area under the | Standard | | interval | |
| the ROC curve | error^{a} | AsympSig.^{b} | Lower | Upper |
| 0.744 | 0.031 | <0.001 | 0.684 | 0.805 |

Notes:
^{a}under non-parametric test;
^{b}null hypothesis: actual area = 0.5.

(4) Diagnosis of Cirrhosis from Chronic Hepatitis B Using the 4210 Da Peptide as a Molecular Marker

The 130 serum samples from the patients with chronic hepatitis B and the 130 serum samples from the patients with cirrhosis were analyzed herein.

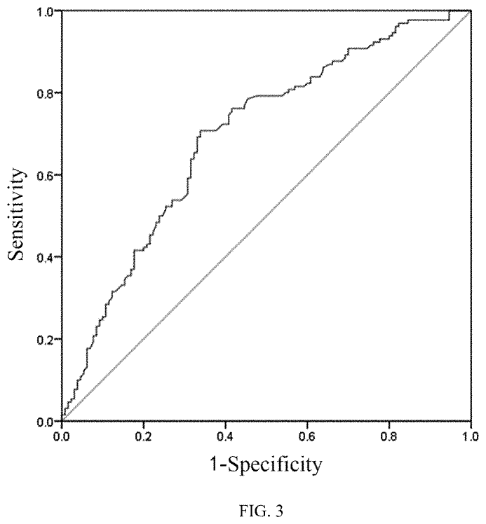

The abundance of the 4210 Da peptide in the serum sample of chronic hepatitis B was used as an indicator, and 232.50 was used as a threshold value, where a case can be determined to suffer from cirrhosis when the abundance of the 4210 Da peptide was less than 232.5. Sensitivity, specificity, Youden index and accuracy of the diagnosis of cirrhosis from chronic hepatitis B were shown in Table 4; the ROC curve was shown in FIG. 3; and the area under the ROC curve was shown in Table 5.

TABLE 4
Sensitivity, specificity, Youden index and accuracy of
the diagnosis of cirrhosis from chronic hepatitis B

| Sensitivity | Specificity | Youden index | Accuracy |
|---|---|---|---|
| 0.662 | 0.708 | 0.369 | 0.685 |

TABLE 5
Area under the ROC curve of the diagnosis
of cirrhosis from chronic hepatitis B

| | | | Asymp 95% confidence | |
|---|---|---|---|---|
| Area under the | Standard | | interval | |
| the ROC curve | error^{a} | AsympSig.^{b} | Lower | Upper |
| 0.695 | 0.033 | <0.001 | 0.631 | 0.759 |

Notes:
^{a}under non-parametric test;
^{b}null hypothesis: actual area = 0.5.

(5) Diagnosis of Hepatocellular Carcinoma from Healthy Control Using the 4210 Da Peptide as a Molecular Marker

The 130 serum samples from the healthy controls and the 130 serum samples from the patients with hepatocellular carcinoma were analyzed herein.

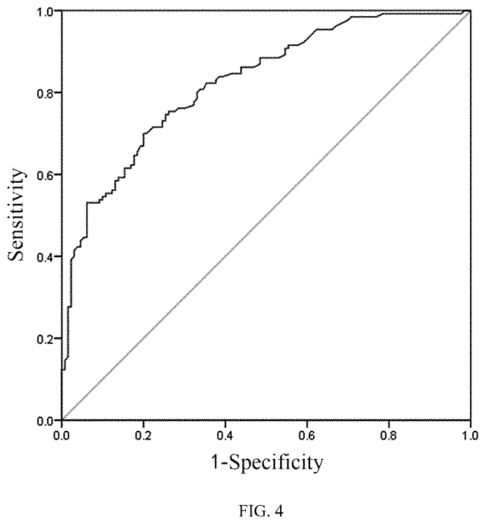

The abundance of the 4210 Da peptide in the serum sample was used as an indicator, and 86.50 was used as a threshold value, where a case can be determined to suffer from hepatocellular carcinoma when the abundance of the 4210 Da peptide was equal to or more than 86.5. Sensitivity, specificity, Youden index and accuracy of the diagnosis of hepatocellular carcinoma from the healthy controls were shown in Table 6; the ROC curve was shown in FIG. 4; and the area under the ROC curve was shown in Table 7.

TABLE 6
Sensitivity, specificity, Youden index and accuracy of the
diagnosis of hepatocellular carcinoma from healthy controls

| Sensitivity | Specificity | Youden index | Accuracy |
|---|---|---|---|
| 0.800 | 0.700 | 0.500 | 0.750 |

TABLE 7
Area under the the ROC curve of the diagnosis of
hepatocellular carcinoma from healthy control

| | | | Asymp 95% confidence | |
|---|---|---|---|---|
| Area under the | Standard | | interval | |
| the ROC curve | error^{a} | AsympSig.^{b} | Lower | Upper |
| 0.824 | 0.025 | <0.001 | 0.775 | 0.874 |

Notes:
^{a}under non-parametric test;
^{b}null hypothesis: actual area = 0.5.

(6) Diagnosis of Cirrhosis from Healthy Controls Using the 4210 Da Peptide as a Molecular Marker

The 130 serum samples from the healthy controls and the 130 serum samples from the patients with cirrhosis were analyzed herein.

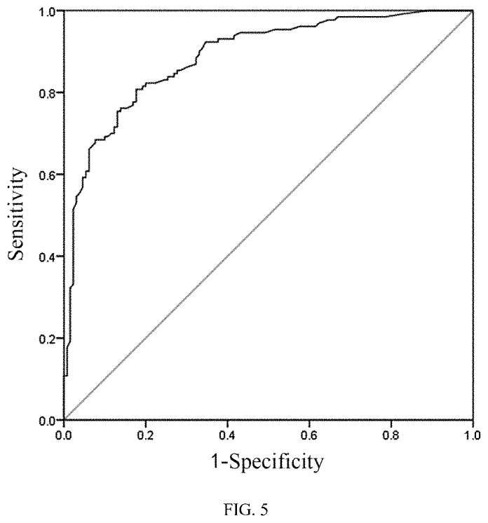

The abundance of the 4210 Da peptide in the serum sample was used as an indicator, and 96.0 was used as a threshold value, where a case can be determined to suffer from cirrhosis when the abundance of the 4210 Da peptide was equal to or more than 96.0. Sensitivity, specificity, Youden index and accuracy of the diagnosis of cirrhosis from the healthy controls were shown in Table 8; the ROC curve was shown in FIG. 5; and the area under the ROC curve was shown in Table 9.

TABLE 8
Sensitivity, specificity, Youden index and accuracy of
the diagnosis of cirrhosis from chronic hepatitis B

| Sensitivity | Specificity | Youden index | Accuracy |
|---|---|---|---|
| 0.823 | 0.808 | 0.631 | 0.816 |

TABLE 7
Area under the the ROC curve of the diagnosis
of cirrhosis from healthy controls

| | | | Asymp 95% confidence | |
|---|---|---|---|---|
| Area under the | Standard | | interval | |
| the ROC curve | error^{a} | AsympSig.^{b} | Lower | Upper |
| 0.888 | 0.020 | <0.001 | 0.848 | 0.928 |

Notes:
^{a}under non-parametric test;
^{b}null hypothesis: actual area = 0.5.

What is claimed is:

1. A method of diagnosing a hepatitis B-associated liver disease in a subject, comprising:
(1) determining an abundance of a 4210 Da peptide in a serum sample from the subject using a matrix-assisted laser desorption ionization time-of-flight mass spectrometer; and
(2) diagnosing the hepatitis B-associated liver disease according to the determined abundance of the 4210 Da peptide in the serum sample.

2. The method of claim 1, wherein in step (2), the diagnosis is performed as follows:
(a1) when 4210 Da peptide is used to discriminate HBV-related cirrhosis/hepatocellular carcinoma from chronic hepatitis, if the abundance of the 4210 Da peptide in the serum sample is greater than or equal to 86.5 and less than 233.5, the hepatitis B-associated liver disease is cirrhosis or hepatocellular carcinoma, otherwise, the hepatitis B-associated liver disease is chronic hepatitis B.

3. The method of claim 1, wherein in step (2), the diagnosis is performed as follows:
(b1) when 4210 Da peptide is used to discriminate HBV-related cirrhosis from normal subject, if the abundance of the 4210 Da peptide in the serum sample is greater than or equal to 96.00, the subject is diagnosed with cirrhosis, otherwise, the subject is a normal subject;
(b2) when 4210 Da peptide is used to discriminate HBV-related hepatocellular carcinoma from normal subject, then if the abundance of the 4210 Da peptide in the serum sample is greater than or equal to 86.5, the subject is diagnosed with hepatocellular carcinoma, otherwise, the subject is normal subject;
(b3) when 4210 Da peptide is used to discriminate HBV-related cirrhosis from chronic hepatitis, if the abundance of the 4210 Da peptide in the serum sample is less than 232.50 and the subject suffers from chronic hepatitis B, the hepatitis B-associated liver disease is cirrhosis, otherwise, the hepatitis B-associated liver disease is chronic hepatitis B; or
(b4) when 4210 Da peptide is used to discriminate HBV-related hepatocellular carcinoma from chronic hepatitis, if the abundance of the 4210 Da peptide in the serum sample is less than 233.5 and the subject suffers from chronic hepatitis B, the hepatitis B-associated liver disease is hepatocellular carcinoma, otherwise, the hepatitis B-associated liver disease is chronic hepatitis B.
